(19) United States
(12) Patent Application Publication
(10) Pub. No.: US 2025/0339398 A1
(43) Pub. Date: Nov. 6, 2025

(54) TREATMENT OF SLEEP DISTRUBANCES IN AUTISM SPECTRUM DISORDER PATIENTS

(71) Applicant: Vanda Pharmaceuticals Inc., Washington, DC (US)

(72) Inventors: Mihael H. Polymeropoulos, Washington, DC (US); Christos Polymeropoulos, Washington, DC (US); Sandra SMIESZEK, Washington, DC (US)

(21) Appl. No.: 18/691,426

(22) PCT Filed: Sep. 14, 2022
(86) PCT No.: PCT/US2022/076393

§ 371 Date: Mar. 12, 2024

Related U.S. Application Data
(60) Provisional application No. 63/243,918, filed on Sep. 14, 2021.
(60) Provisional application No. 63/268,430, filed on Feb. 23, 2022.
(60) Provisional application No. 63/269,137, filed on Mar. 10, 2022.

(51) Int. Cl. A61K 31/343; C12Q 1/6883

(52) U.S. Cl. CPC A61K 31/343; C12Q 1/6883; C12Q 2600/106; C12Q 2600/156

(57) ABSTRACT

The invention relates generally to the treatment of autism spectrum disorder (ASD) and sleep disturbances and, more particularly, to the treatment of sleep disorders and/or the improvement of daytime functioning in ASD patients using tasimelteon.

### CROSS-REFERENCE TO RELATED APPLICATIONS

This application claims priority to U.S. Provisional Patent Application Ser. No. 63/243,918, filed 14 Sep. 2021, U.S. Provisional Patent Application Ser. No. 63/268,430, filed 23 Feb. 2022, and U.S. Provisional Patent Application Ser. No. 63/269,137, filed 10 Mar. 2022, the entirety of each of which is hereby incorporated herein as though fully set forth.

### BACKGROUND

### Autism Spectrum Disorders

Autism spectrum disorders (ASDs) are neurodevelopmental disorders associated with persistent deficits in social communication and restricted/repetitive behaviors, among other criteria. ASDs consist of a wide range of presentations and levels of severity requiring minimal to substantial support.

Symptoms of ASDs include those affecting social communication and interaction as well as restricted or repetitive behaviors or interests. Known symptoms of ASDs include, for example, avoidance of or inability to maintain eye contact; lack of facial expression; reduced use of gestures, including hand gestures; decreased sharing of interests with others; failure to point or look where others point; failure to notice when others are hurt or sad; failure to pretend in play; lack of interest in peers; difficulty understanding others' feelings or discussing their own feelings; failure to play games that involve turn-taking; lining up objects when upset or object order is changed; repetition of words or phrases (echolalia); playing with toys the same way every time; focusing on parts of objects; becoming upset by minor changes; obsessive interests; unusual adherence to routines; flapping of hands; body rocking; spinning in circles; unusual reactions to sounds, smells, tastes, looks, or feels; delayed language skills; delayed movement skills; delayed cognitive or learning skills; hyperactive, impulsive, and/or inattentive behavior; epilepsy or seizure disorder; unusual eating or sleeping habits; sleep disturbances or impaired daytime functioning; gastrointestinal issues; unusual mood or emotional reactions; anxiety, stress, or excessive worry; and a lack of fear or more fear than expected.

Sleep disruption in individuals with ASDs has a documented prevalence of up to 80%, higher than in healthy control subjects. Sleep complaints in ASD are varied. However, difficulty falling asleep and restless/nighttime awakening have repeatedly emerged as primary sleep-related issues for individuals with ASD. One or more such sleep complaints may lead to reduced sleep time with attendant consequences in daytime functioning, including worsening of other ASD symptoms. Most sleep related research in ASD has focused on children, but a growing body of research seems to indicate that sleep in adults with ASD is similarly disrupted.

The etiology of sleep disorders in ASD is still an active area of research, but there is evidence suggesting that circadian abnormalities may be a significant source of sleep-related symptoms in ASD. Serum melatonin levels are markedly decreased in ASD participants as compared to healthy controls.

Recent evidence suggests a genetic cause for these abnormalities with disruption of the serotonin-N-acetylserotonin-melatonin pathway with polymorphisms identified in the acetylserotonin O-methyltransferase (ASMT) gene. In addition to abnormal levels of melatonin detected in individuals with ASD, mutations in regulatory genes controlling the expression of melatonin receptors MTNR1A and MTNR1B are known. Taken together, these studies suggest that melatonin synthesis and signaling is aberrant in ASD and may be a causal factor in producing sleep-related symptoms.

At the present time, there is no approved treatment for the sleep symptoms in ASD.

### Smith-Magenis Syndrome (SMS)

Smith-Magenis Syndrome (SMS) is caused by an interstitial deletion of 17p11.2 or, more rarely, a variant in the retinoic acid induced 1 (RAI1) gene. The prevalence of SMS is estimated to be between 1 in 15,000 and 1 in 25,000. Haploinsufficiency of RAI1 is the primary cause of the neurobehavioral and metabolic phenotype in SMS. Individuals with SMS present with a distinct pattern of mild to moderate intellectual disability, delayed speech and language skills, distinctive craniofacial and skeletal abnormalities, behavioral disturbances, and, almost uniformly, significant sleep disturbances. Alterations in RAI1 copy number have also been linked to a number of neurodevelopmental disorders, including ASD.

### Tasimelteon

As a circadian regulator, tasimelteon is a dual melatonin receptor agonist approved in the United States under the trade name HETLIOZ® for the treatment of Non-24-Hour Sleep-Wake Disorder (Non-24) and for the treatment of Nighttime Sleep Disturbances in Smith-Magenis Syndrome (SMS). Tasimelteon was shown to be safe and well-tolerated in previous clinical studies in healthy volunteers, in participants with primary insomnia, in participants with Non-24, and in pediatric participants with SMS.

Tasimelteon has a greater affinity for the MT2 receptor than the MT1 receptor. For doses ranging from 3 mg to 300 mg, the pharmacokinetics of tasimelteon are linear. Tasimelteon has an absolute bioavailability of 38% and its peak plasma concentration (tₘₐₓ) occurs approximately 30 minutes to three hours after fasted oral administration. Liquid formulations of tasimelteon and methods for their administration are described in International Patent Application Publication No. WO/2021/119456, which is hereby incorporated herein as though fully set forth.

### SUMMARY

In one embodiment, the invention provides a method of treating a patient suffering from autism spectrum disorder (ASD), the method comprising: administering to the patient a dose of tasimelteon effective to improve at least one symptom of ASD.

In another embodiment, the invention provides a method of treating a patient suffering from a sleep disturbance associated with autism spectrum disorder (ASD), the method comprising: administering to the patient a dose of tasimelteon effective to improve sleep, daytime functioning, or both.

In still another embodiment, the invention provides, in a method of improving sleep in patients consisting essentially of administering to said patients an amount of tasimelteon effective to improve sleep, the improvement comprising: selecting a patient for said treatment by identifying that said patient suffers from a sleep disturbance associated with autism spectrum disorder (ASD).

In one embodiment, the invention provides a method of treating a patient suffering from autism spectrum disorder (ASD), the method comprising: determining or otherwise identifying that the patient carries one or more variant in the retinoic acid induced 1 (RAI1) gene associated with reduced RAI1 function; and in the case that the patient carries one or more said variant, administering to the patient a dose of tasimelteon effective to reduce a sleep disturbance experienced by the patient.

In another embodiment, the invention provides, in a method of treating a sleep disturbance in a patient, an improvement comprising: selecting as said patient an individual carrying at least one variant in the retinoic acid induced 1 (RAI1) gene associated with reduced RAI1 function.

### DETAILED DESCRIPTION

In a study to assess the efficacy of tasimelteon in treating sleep disturbances in ASD, patients selected for treatment based on an ASD diagnosis and a recent history of sleep disturbance defined as a minimum 45-minute sleep latency on at least three nights per week and continuing for at least three months. Those patients for whom the sleep disturbance may be attributed to another diagnosable disorder or medication are excluded, as are those patients who test positive for or have symptoms suggestive of sleep apnea.

A large scale association analysis of the ASD MSSNG database reveals a number of findings potentially relevant to the treatment of ASD. The ASD MSSNG database includes the genomes of over 11,000 individuals representing over 4,000 families. Approximately half of the individuals represented in the ASD MSSNG database are affected with ASD.

The analysis reveals a single case of the 17p11.2 deletion known to cause SMS and three additional microdeletions in RAI1 exon 3. In addition, the analysis reveals 53 RAI1 missense variants, two frameshift variants, and one splicing variant.

Table 1 below shows the details of each of these missense, frameshift, and splicing variants, including the start and end positions; wildtype or reference allele and alternate allele; details of the variant location within the RAI1 gene and resulting change in protein sequence, where applicable; and predicted impact in terms of RAI1 function. All references to start and end positions and the wildtype or reference allele are with respect to RAI1 transcript NM 030665.3 (available at https://databases.lovd.nl/shared/refseq/RAI1_NM_030665.3_codingDNA.html).

TABLE 1
RAI variants identified in ASD MSSNG database

| Start | End | Ref. | Alt. | Variant | Effect - Impact |
|---|---|---|---|---|---|
| 17793391 | 17793393 | GA | AGC | exon3:c.444_445delinsAGC | Frameshift-High, Stop |
| | | | | | Gain-High |
| 17795742 | 17795743 | T | C | exon3:c.T2795C:p.V932A | Missense-High |
| 17793773 | 17793774 | T | C | exon3:c.T826C:p.Y276H | Missense-High |
| 17793990 | 17793991 | G | A | exon3:c.G1043A:p.R348H | Missense-High |
| 17795495 | 17795496 | T | C | exon3:c.T2548C:p.F850L | Missense-High |
| 17798082 | 17798083 | T | A | exon3:c.T5135A:p.L1712H | Missense-High |
| 17793048 | 17793049 | G | A | exon3:c.G101A:p.S34N | Missense-High |
| 17793193 | 17793194 | C | G | exon3:c.C246G:p.S82R | Missense-High |
| 17794415 | 17794416 | G | A | exon3:c.G1468A:p.A490T | Missense-High |
| 17794722 | 17794723 | G | C | exon3:c.G1775C:p.R592P | Missense-High |
| 17794784 | 17794785 | G | A | exon3:c.G1837A:p.G613R | Missense-High |
| 17796491 | 17796492 | C | T | exon3:c.C3544T:p.H1182Y | Missense-High |
| 17793078 | 17793079 | G | A | exon3:c.G131A:p.R44Q | Missense-High |
| 17793258 | 17793259 | A | G | exon3:c.A311G:p.D104G | Missense-High |
| 17793435 | 17793436 | A | G | exon3:c.A488G:p.Q163R | Missense-High |
| 17793729 | 17793730 | A | G | exon3:c.A782G:p.Q261R | Missense-High |
| 17793890 | 17793891 | G | A | exon3:c.G943A:p.G315R | Missense-High |
| 17794257 | 17794258 | C | G | exon3:c.C1310G:p.A437G | Missense-High |
| 17794505 | 17794506 | G | A | exon3:c.G1558A:p.G520S | Missense-High |
| 17794526 | 17794527 | C | T | exon3:c.C1579T:p.R527C | Missense-High |
| 17794829 | 17794830 | G | A | exon3:c.G1882A:p.E628K | Missense-High |
| 17794872 | 17794873 | C | T | exon3:c.C1925T:p.S642L | Missense-High |
| 17795682 | 17795683 | C | T | exon3:c.C2735T:p.A912V | Missense-High |
| 17683704 | 17683705 | T | C | RAI1:splicing | Predicted Splicing-High |
| 17793297 | 17793298 | G | C | exon3:c.G350C:p.S117T | Missense-High |
| 17794797 | 17794798 | C | A | exon3:c.C1850A:p.A617D | Missense-High |
| 17793068 | 17793069 | G | A | exon3:c.G121A:p.D41N | Missense-High |
| 17793929 | 17793930 | G | A | exon3:c.G982A:p.V3281 | Missense-High |
| 17794280 | 17794281 | A | G | exon3:c.A1333G:p.1445V | Missense-High |
| 17796155 | 17796156 | G | A | exon3:c.G3208A:p.G1070R | Missense-High |
| 17796609 | 17796610 | C | A | exon3:c.C3662A:p.A1221E | Missense-High |
| 17797196 | 17797197 | A | G | exon3:c.A4249G:p.K1417E | Missense-High |
| 17797557 | 17797558 | G | A | exon3:c.G4610A:p.R1537Q | Missense-High |
| 17798435 | 17798436 | G | A | exon3:c.G5488A:p.V1830M | Missense-High |
| 17803842 | 17803843 | G | A | exon4:c.G5653A:p.D1885N | Missense-High |
| 17803779 | 17803780 | G | A | exon4:c.G5590A:p.G1864R | Missense-High, |
| | | | | | Predicted Splicing- |
| | | | | | Medium |
| 17798167 | 17798168 | C | CA | exon3:c.5221dupA:p.L1740fs | Frameshift-High |
| 17794563 | 17794564 | C | T | exon3:c.C1616T:p.P539L | Missense-High |
| 17795172 | 17795173 | A | G | exon3:c.A2225G:p.N742S | Missense-High |
| 17796143 | 17796144 | C | A | exon3:c.C3196A:p.P1066T | Missense-High |
| 17796428 | 17796429 | C | T | exon3:c.C3481T:p.R1161W | Missense-High |
| 17793105 | 17793106 | C | T | exon3:c.C158T:p.P53L | Missense-High |
| 17793666 | 17793667 | C | T | exon3:c.C719T:p.T2401 | Missense-High |
| 17793918 | 17793919 | C | T | exon3:c.C971T:p.P324L | Missense-High |
| 17793986 | 17793987 | G | A | exon3:c.G1039A:p.A347T | Missense-High |
| 17794434 | 17794435 | C | T | exon3:c.C1487T:p.P496L | Missense-High |
| 17794734 | 17794735 | G | A | exon3:c.G1787A:p.R596Q | Missense-High |
| 17794851 | 17794852 | A | T | exon3:c.A1904T:p.D635V | Missense-High |
| 17794995 | 17794996 | G | C | exon3:c.G2048C:p.S683T | Missense-High |
| 17795541 | 17795542 | T | C | exon3:c.T2594C:p.L865P | Missense-High |
| 17795886 | 17795887 | C | A | exon3:c.C2939A:p.A980D | Missense-High |
| 17795906 | 17795907 | G | A | exon3:c.G2959A:p.A987T | Missense-High |
| 17796552 | 17796553 | G | A | exon3:c.G3605A:p.R1202Q | Missense-High |
| 17796596 | 17796597 | C | T | exon3:c.C3649T:p.R1217W | Missense-High |
| 17797293 | 17797294 | G | A | exon3:c.G4346A:p.R1449Q | Missense-High |
| 17798117 | 17798118 | C | T | exon3:c.C5170T:p.R1724W | Missense-High |

The presence of one or more of the variants of Table 1 in the genome of an ASD patient is predicted to result in some reduction in function of the RAI1 gene, up to and including a loss of function of the gene. A patient is deemed to carry one or more of the variants of Table 1 if that patient is heterozygous, homozygous, or compound heterozygous for any of the variants of Table 1.

As noted above, haploinsufficiency of RAI1 is the primary cause of neurobehavioral and metabolic symptoms in SMS patients, with significant sleep disturbances being the most common symptom. The current prevailing theory is that there is an underlying circadian pathophysiology causing sleep disturbances in SMS associated with RAI1 haploinsufficiency. These patients exhibit low overall melatonin concentrations and abnormal timing of peak plasma melatonin concentrations. This abnormal melatonin rhythm is estimated to occur in 95% of SMS patients.

However, both ASD and SMS patients suffer from sleep disturbances. The variation of sleep disturbances seen in SMS patients may overlap in ASD patients, especially in those with consequential variants in the RAI1 gene such as those identified in Table 1 or the deletion of 17p11.2.

Accordingly, embodiments of the invention are directed to the treatment of sleep disturbances in ASD patients carrying one or more variant associated with reduced RAI1 function. The treatment of a sleep disturbance may be assessed directly and/or in terms of improvement in daytime functioning, as will be described further below.

According to some embodiments, ASD patients determined to carry one or more such variant are administered tasimelteon orally once daily approximately one hour before bedtime. In all cases, tasimelteon is administered under fasted conditions, which includes the administration of tasimelteon without food and the administration of tasimelteon following a period during which the patient has ingested no food. The administration of tasimelteon under fasted conditions is described in U.S. Pat. No. 10,376,487, which is hereby incorporated herein as though fully set forth.

Dosing is based on age and/or body mass. Adult patients and pediatric patients having a body mass of 28 kg or more are administered 20 mg of tasimelteon in either capsule or oral suspension (4 mg/mL) form. Pediatric patients having a body mass of less than 28 kg are administered 0.7 mg/kg of tasimelteon in oral suspension (4 mg/mL) form. Patients aged 16 or 17 having a body mass of 28 kg or more are administered either the 20 mg capsule or the 4 mg/mL oral suspension, at the patient's choice.

Efficacy of treatment with tasimelteon, in terms of improvement in sleep disturbances and/or improved daytime functioning, is assessed using one or more known scales: the Patient Global Impression—Change (PGI-C) scale, the Patient Global Impression—Change Behavior (PGI-C Behavior) scale, the Clinical Global Impression—Severity (CGI-S) scale, the Clinical Global Impression—Change (CGI-C) scale, a Daily eDiary, the Aberrant Behavior Checklist (ABC).

The PGI-C is a 7-point rating scale where participants or their guardians rate the participant's improvement in symptoms relative to the start of the study. It is rated as: 1. Very much improved, 2. Much improved, 3. Minimally improved, 4. No change, 5. Minimally worse, 6. Much worse, or 7. Very much worse.

The PGI-C Behavior is a similar 7-point rating scale where participants or their guardians rate the participant's improvement in behavioral issues relative to the start of the study.

The CGI-S is a similar 7-point rating scale where investigators rate the severity of a participant's condition at the time of assessment, relative to the clinician's past experience with participants who have the same diagnosis. It is rated as: 1. Normal not at all ill, 2. Borderline ill, 3. Mildly ill, 4. Moderately ill, 5. Markedly ill, 6. Severely ill, or 7. Extremely ill.

The CGI-C is a similar 7-point rating scale where investigators rate a participant's improvement in symptoms relative to the start of the study. It is rated as: 1. Very much improved, 2. Much improved, 3. Minimally improved, 4. No change, 5. Minimally worse, 6. Much worse, or 7. Very much worse.

Daily eDiaries consist of questionnaires that a participant or caregiver fill out daily and comprise sleep diaries and additional questionnaires such as post-and pre-sleep questionnaires that report, for example, a participant's sleep onset and wake time, an assessment of nighttime sleep, daytime sleep episodes, etc. These questionnaires may include the PGI-S Behavior scale, a 5-point rating scale where participants or their guardians rate the severity of the participant's behavior issues at the time of assessment. It is rated as: 0. None, 1. Mild, 2. Moderate, 3. Severe, or 4. Very severe.

Sleep diaries, such as the Daily eDiaries above, may be used to assess various sleep parameters before, during, and/or after treatment according to the invention, as well as changes in such parameters in response to treatment. For example, sleep onset and wake time may be used to measure sleep time before, during, and/or after treatment according to the invention.

The ABC is a validated symptom checklist for parents and caregivers to assess problem behaviors of children and adults. Its 58 items resolve into five subscales: (1) Irritability, Agitation, (2) Lethargy, Social Withdrawal, (3) Stereotypic Behavior, (4) Hyperactivity, Noncompliance, and (5) Inappropriate Speech.

Other methods of assessing efficacy of treatment may be employed, as will be recognized by one skilled in the art, and are within the scope of the invention. Improved sleep may include a reduction in sleep onset latency (SOL).

As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

This written description uses examples to disclose the invention, including the best mode, and also to enable any person skilled in the art to practice the invention, including making and using any devices or systems and performing any related or incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A method of treating a patient suffering from autism spectrum disorder (ASD), the method comprising:
administering to the patient a dose of tasimelteon effective to improve at least one symptom of ASD.

2. The method of claim 1, wherein the at least one symptom of ASD includes a symptom affecting social communication or interaction selected from a group consisting of: avoidance of or inability to maintain eye contact; lack of facial expression; reduced use of gestures, including hand gestures; decreased sharing of interests with others; failure to point or look where others point; failure to notice when others are hurt or sad; failure to pretend in play; lack of interest in peers; difficulty understanding others' feelings or discussing the patient's own feelings; and failure to play games that involve turn-taking.

3. (canceled)

4. The method of claim 1, wherein the at least one symptom includes a symptom affecting restricted or repetitive behaviors or interests selected from a group consisting of: lining up objects when upset or object order is changed; repetition of words or phrases (echolalia); playing with toys the same way every time; focusing on parts of objects; becoming upset by minor changes; obsessive interests; unusual adherence to routines; flapping of hands; body rocking; spinning in circles; and unusual reactions to sounds, smells, tastes, looks, or feels.

5. (canceled)

6. The method of claim 1, wherein the at least one symptom is selected from a group consisting of: delayed language skills; delayed movement skills; delayed cognitive or learning skills; hyperactive, impulsive, and/or inattentive behavior; epilepsy or seizure disorder; unusual eating or sleeping habits; sleep disturbances or impaired daytime functioning; gastrointestinal issues; unusual mood or emotional reactions;
anxiety, stress, or excessive worry; and a lack of fear or more fear than expected.

7. The method of claim 1, wherein improvement of the at least one symptom includes an improvement in at least one measure selected from a group consisting of: clinical global impression of change (CGI-C) scale, clinical global impression of severity (CGI-S) scale, patient global impression of change (PGI-C) scale, aberrant behavior checklist (ABC), patient global impression of severity behavior (PGI-S Behavior) scale, and patient global impression of change behavior (PGI-C Behavior) scale.

8. The method of claim 1, wherein the patient suffers from a sleep disturbance associated with autism spectrum disorder (ASD) and the dose of tasimelteon is effective to improve sleep, daytime functioning, or both,
wherein improved sleep includes an improvement in at least one measure selected from a group consisting of: sleep onset latency (SOL), clinical global impression of change (CGI-C) scale, clinical global impression of severity (CGI-S) scale, and patient global impression of change (PGI-C) scale; and
improved daytime functioning includes an improvement in at least one measure selected from a group consisting of: aberrant behavior checklist (ABC), patient global impression of severity behavior (PGI-S Behavior) scale, and patient global impression of change behavior (PGI-C Behavior) scale.

9-11. (canceled)

12. The method of claim 8, wherein the sleep disturbance includes sleep latency of at least 45 minutes.

13. The method of claim 8, wherein the dose of tasimelteon is 20 mg.

14. The method of claim 13, wherein administering includes administering the tasimelteon once daily before bedtime.

15. The method of claim 14, wherein administering includes administering the tasimelteon orally without food.

16. The method of claim 8, wherein the dose of tasimelteon is 0.7 mg/kg.

17. (canceled)

18. (canceled)

19. In a method of improving sleep in patients consisting essentially of administering to said patients an amount of tasimelteon effective to improve sleep, the improvement comprising:
selecting a patient for said treatment by identifying that said patient suffers from a sleep disturbance associated with autism spectrum disorder (ASD).

20. The improvement of claim 19, wherein improved sleep includes an improvement in at least one measure selected from a group consisting of:
sleep onset latency (SOL), clinical global impression of change (CGI-C) scale, clinical global impression of severity (CGI-S) scale, and patient global impression of change (PGI-C) scale.

21. (canceled)

22. The improvement of claim 20-21, wherein the sleep disturbance includes sleep latency of at least 45 minutes.

23. The improvement of claim 19, wherein the amount of tasimelteon is 20 mg.

24. The improvement of claim 23, wherein administering includes administering the tasimelteon once daily before bedtime.

25. The improvement of claim 24, wherein administering includes administering the tasimelteon orally without food.

26. The improvement of claim 19, wherein the amount of tasimelteon is 0.7 mg/kg.

27. (canceled)

28. (canceled)

29. A method of treating a patient suffering from autism spectrum disorder (ASD), the method comprising:
determining or otherwise identifying that the patient carries one or more variant in the retinoic acid induced 1 (RAI1) gene associated with reduced RAI1 function; and
in the case that the patient carries one or more said variant, administering to the patient a dose of tasimelteon effective to reduce a sleep disturbance experienced by the patient,
wherein the one or more variant in the RAI1 gene is selected from a group consisting of: the variants of Table 1 and a deletion of 17p11.2.

30. (canceled)

31. The method of claim 29, wherein the one or more variant includes a frameshift variant.

32. The method of claim 31, wherein the frameshift variant is a GA-to-AGC mutation at positions 444 and 445 of the RAI1 sequence.

33. The method of claim 31, wherein the frameshift variant is a C-to-CA mutation at position 5221 of the RAI1 sequence.

34. The method of claim 29, wherein the sleep disturbance is reduced sleep time and the amount of tasimelteon is effective to increase sleep time in the patient.

35. In a method of treating a patient suffering from a sleep disturbance, the improvement comprising:
selecting as said patient an individual carrying at least one variant in the retinoic acid induced 1 (RAI1) gene associated with reduced RAI1 function,
wherein said at least one variant is selected from a group consisting of: the variants of Table 1 and a deletion of 17p11.2.

36. (canceled)

37. The improvement of claim 35, wherein the one or more variant includes a frameshift variant.

38. The improvement of claim 37, wherein the frameshift variant is a GA-to-AGC mutation at positions 444 and 445 of the RAI1 sequence.

39. The improvement of claim 37, wherein the frameshift variant is a C-to-CA mutation at position 5221 of the RAI1 sequence.

40. The improvement of claim 35, wherein said patient suffers from autism spectrum disorder (ASD).

41. The improvement of claim 35, wherein said patient suffers from Smith-Magenis Syndrome (SMS).

42. The improvement of claim 35, wherein the sleep disturbance is reduced sleep time.
